(19) United States
(12) Patent Application Publication
(10) Pub. No.: US 2010/0183710 A1
(43) Pub. Date: Jul. 22, 2010

(54) OMEPRAZOLE FORM B

(76) Inventors: Vijayabhaskar Bolugoddu, Hyderabad (IN); Jaydeepkumar Dahyabhai Lilakar, Gujarat (IN); Ramchandra Reddy Pingili, Hyderabad (IN); Swarupa Reddy Dudipala, Hyderabad (IN); Adolf Ceasor Goldwyn, Hyderabad (IN); Bala Murali Krishna Pittala, Guntur District (IN); Mailatur Sivaraman Mohan, Hyderabad (IN); Indu Bhushan, Hyderabad (IN); Ravinder Kodipyaka, Kaghaznagar (IN); Rahul Sudhakar Gawande, Maharashtra (IN); Srikanth Basety, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 11/994,943

(22) PCT Filed: Jul. 7, 2006
(86) PCT No.: PCT/US06/26333

§ 371 Date: Jan. 7, 2008

Related U.S. Application Data
(60) Provisional application No. 60/734,349, filed on Nov. 7, 2005.

(30) Foreign Application Priority Data
Jul. 7, 2005 (IN) 894/CHE/2005

(51) Int. Cl. A61K 9/64; C07D 401/12; A61K 31/4439; A61K 9/16

(52) U.S. Cl. 424/456; 546/273.7; 514/338; 424/493

(57) ABSTRACT

A process for preparing pure omeprazole Form B, and compositions containing omeprazole Form B.

### INTRODUCTION TO THE INVENTION

The present invention relates to a process for the preparation of crystalline Form B of omeprazole.

Chemically, omeprazole is 5-Methoxy-2-[[(4-methoxy-3,5-dimethyl-2-pyridinyl)methyl]sulfinyl]-1H-benzimidazole and has the structural formula as shown in Formula I.

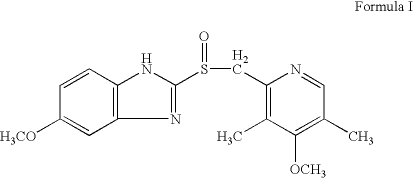

Omeprazole is available commercially in products sold as ZEGERID™ powder for oral suspension in unit dose packets as an immediate release formulation, each packet containing 40 or 20 mg of omeprazole. It is also available as PRILOSEC™ delayed release capsules containing 10, 20, or 40 mg of omeprazole. These products are indicated for the treatment of gastric ulcers, gastroesophageal reflux disease, and for short-term treatment of active duodenal ulcers.

Omeprazole and its salts were described in European Patent No. 5129, and in U.S. Pat. Nos. 4,255,431, 4,508,905, and 4,337,257.

The single crystal X-ray data and the derived molecular structure of a crystalline form of omeprazole were described by Ohishi et al., Acta Cryst. (1989), C45, 1921-1923. This crystal form of omeprazole was designated as Form B by later publications.

U.S. Pat. No. 6,150,380 describes crystalline Form A of omeprazole and processes for the preparation of Form A and Form B. The patent also describes that omeprazole Form A is well-defined, thermodynamically more stable and less hygroscopic than omeprazole Form B. The aforesaid patent also describes that omeprazole Form B can completely or partially convert into Form A.

U.S. Patent Application Publication No. US 2006/0079560 describes crystalline Form C of omeprazole and a process for its preparation.

There is a continuing need to prepare stable crystalline forms of active substances such as omeprazole in an industrially simple and readily feasible way with high yields.

### SUMMARY OF THE INVENTION

The present invention relates to a process for the preparation of crystalline Form B of omeprazole.

In an embodiment, a process for preparation of omeprazole Form B comprises the steps of:

a) providing a solution of omeprazole in a suitable solvent;

b) washing the solution obtained in step a) with water;

c) removing solvent from the solution of step b); and optionally

c) further drying the solid of step (c) to afford crystalline Form B of omeprazole.

Another embodiment of the invention provides a pharmaceutical composition comprising crystalline omeprazole Form B and at least one pharmaceutically acceptable excipient. Such pharmaceutical composition may be administered to a mammalian patient in a dosage form.

An aspect of the invention includes a process for preparing omeprazole crystalline Form B, comprising removing solvents from a solution of omeprazole in a mixture comprising a halogenated hydrocarbon and an alcohol.

Another aspect of the invention includes a composition comprising omeprazole crystalline Form B, coated onto a pharmaceutically inert core.

A further aspect of the invention includes a composition comprising omeprazole crystalline Form B, coated onto a pharmaceutically inert core, and having an outer enteric coating.

### BRIEF DESCRIPTION OF THE DRAWINGS

FIG. 1 is an X-ray power diffraction (“XRPD”) pattern of crystalline Form B of omeprazole prepared in Example 1.

FIG. 2 is an infrared (“IR”) absorption spectrum of crystalline Form B of omeprazole prepared in Example 1.

FIG. 3 is a differential scanning calorimetric (“DSC”) curve of crystalline Form B of omeprazole prepared in Example 1.

FIG. 4 shows a thermogravimetric analysis (“TGA”) curve of crystalline Form B of omeprazole, prepared in Example 1.

FIG. 5 is an XRPD pattern of Form B of omeprazole that contains 5 percent w/w of Form A.

FIG. 6 is a quantification curve using XRPD for substantially pure omeprazole Form B obtained in Example 1.

### DETAILED DESCRIPTION OF THE INVENTION

The present invention relates to a process for the preparation of crystalline Form B of omeprazole.

In an embodiment a process for the preparation of omeprazole Form B comprises the steps of:

a) providing a solution of omeprazole in a suitable solvent;

b) washing the solution obtained in step a) with water;

c) removing the solvent from solution of step b); and optionally

d) further drying the solid obtained in step (c) to afford crystalline Form B of omeprazole.

Step a) involves providing a solution of omeprazole in a suitable solvent

The solution of omeprazole may be obtained by dissolving omeprazole in a suitable solvent, or such a solution may be obtained directly from a reaction in which omeprazole is formed.

When the solution is prepared by dissolving omeprazole in a suitable solvent, any form of omeprazole such as any crystalline or amorphous form, including any salts, solvates and hydrates may be utilized for preparing the solution.

Suitable solvents which can be used in step a) include: alcohols such as methanol, ethanol, isopropyl alcohol, n-propanol, and the like; ketones such as acetone, ethyl methyl ketone, methyl isobutyl ketone and the like; halogenated hydrocarbons such as dichloromethane, 1,2-dichloroethane, chloroform, carbon tetrachloride and the like; or mixtures thereof in various proportions.

When a mixture of solvents is used, the solvents may be chosen from different classes and usually the mixture of solvents contains a water miscible and a water immiscible solvent. In one embodiment, a mixture of dichloromethane and methanol is used for solution preparation, in a volume ratio of about 4:1.

The concentration of the solution can be about 0.1 g/ml of the solvent to 20 g/ml of the solvent or more. Any other concentration may be used as long as a clear solution is obtained.

The order of charging different materials is not critical for the quality of product obtained. A specific order may be preferred with respect to the equipment actually used and will be easily determined by a person skilled in the art.

Suitable temperatures for preparing the solution of omeprazole may range form about 0 to 20° C., or about 0 to 40° C. Since omeprazole and its solution are sensitive to higher temperatures, the solution is preferably prepared at lower temperatures.

Step b) involves washing the solution obtained in step a) with water;

Omeprazole solution obtained in step a) is washed with water to remove any of the water soluble components present in the solution.

The quantity of water used for washing may range from about 1 to 5 times, or about 2 times, the weight of omeprazole present in the solution. Optionally, the solution obtained after water washing may be filtered to remove the undissolved particles.

The undissolved particles can be removed suitably by filtration, centrifugation, and other techniques. The solution can be filtered by passing through paper, glass fiber, or other membrane material, or a clarifying agent such as celite. Depending upon the equipment used and the concentration and temperature of the solution, the filtration apparatus may be preheated to avoid premature crystallization.

Step c) involves removing the solvent from the solution of step b) using a suitable technique. The evaporation proceeds sufficiently rapidly to form the desired product.

Suitable techniques which can be used for the evaporation of the solvent include, but are not limited to agitated thin film evaporators (“ATFD”), suppressed boiling type evaporation, and others which use flash evaporation techniques.

These techniques are applicable to solutions of omeprazole in any high boiling or low boiling solvents. However, solutions using the more volatile organic solvents are preferred. In brief, the process involves drying of a solution of omeprazole on a heated, constantly moving surface under vacuum.

Other techniques such as Buchi Rotavapor drying and drying under vacuum may also be used. An embodiment of the invention involves the evaporation of the solvent using agitated thin film drying-vertical (“ATFD-V”) equipment.

The ATFD-V technique uses high vacuum along with elevated temperatures, which allows operation at lower temperatures. This allows for a short residence time for the product in the drier, which is an important feature for a heat-sensitive product like omeprazole. The solvent evaporation can be achieved in a single pass, avoiding product recirculation and possible degradation. It is suitable for viscous products, and the operating pressures are from atmospheric down to 1 mbar. The equipment can be operated at a wide range of temperatures, such as 25 to 350° C. or more.

The concentration, solvent type, temperature, vacuum, and feeding rate are set at conditions where the omeprazole coming from the inlet precipitates virtually instantly without being exposed to elevated temperatures for extended periods of time.

The process is carried out at lower temperatures of about 35° C. to about 50° C. under reduced pressure of about 600 to about 700 mm Hg. These dryers are indirectly heated and therefore air does not come in contact with the product.

The solution of omeprazole may be added dropwise or continuously to the drying chamber. The speed of the addition of the solution will depend on the solvent used, the viscosity of the mixture, and the height of the chamber. The rate of flow may range from 45 to 65 liters/hour. These and other parameters are well known to a person skilled in the art of evaporation using ATFD.

The drop of solution evaporates almost instantaneously in the chamber. The solidification is spontaneous and does not require further action such as stirring. This instant evaporation allows for obtaining a phase change (solidification) before the solution contacts the bottom of an industrial sized chamber when fed from the top.

ATFD-V helps in evaporating solvents by using heat transfer across the walls and prevents the growth of crystals and particles that can trap the solvent at higher levels. The resulting omeprazole is a crystalline form having a residual solvent content lower than the compound obtained from other techniques of evaporation like the Buchi Rotavapor.

The yields obtained using this technique are superior to those obtained using other techniques.

Step d) involves further drying of the isolated product of step c) to afford the crystalline Form B of omeprazole.

Drying can be suitably carried out in a tray dryer, vacuum oven, air oven, or using a fluidized bed drier, spin flash dryer, flash dryer and the like.

Drying can be carried out under reduced pressure until the residual solvent content reduces to within the limits given by the ICH guidelines. The solvent level depends on the type of solvent but is not more than about 5000 ppm, or about 4000 ppm, or about 3000 ppm.

The drying can be carried out at reduced pressures, such as below 700 mm Hg or below 50 mm Hg, at temperatures of about 35° C. to about 70° C. The drying can be carried out for any desired time periods, times about 1 to 20 hours being suitable for preparing some products.

Drying can be carried out either continuously or discontinuously, by milling it intermittently.

Drying is more efficient when the particle size of the material is smaller and the surface area is higher, hence milling can be performed prior to the drying operation.

For performing the milling or micronization operation before drying, the material should initially be semi-dried to make it suitable for milling operation. Milling of the wet materials leads to loss in the yields.

Hence for performing the drying operation intermittently, the product is initially dried for a short period of time to get the semi-dried material. Then the semi-dried product can be milled or micronized to get the required particle size. This operation reduces the size of particles and increases surface area of particles by colliding particles with each other at high speeds.

Milling can be done suitably using jet milling equipment like an air jet miller, or using other conventional milling equipment to get the desired particle size suitable for preparing pharmaceutical compositions.

The process of the present invention provides a stable crystalline Form B of omeprazole.

The term stable crystalline form refers to stability of the crystalline form under the standard conditions of testing of pharmaceutical products, wherein the stability is evaluated by stability of the polymorphic form.

Omeprazole crystalline Form B obtained in this invention is substantially free of omeprazole crystalline Form A and it does not convert into crystalline Form A during storage in a container for 6 months at 40° C. and 60% relative humidity.

The samples were analyzed using X-ray crystallography, Omeprazole Form B of present invention is also characterized by a low amount of residual solvents. Omeprazole Form B of present invention has a residual solvent content that is within the limits given by the ICH (International Conference for Harmonzation) guidelines.

Crystalline Form B of omeprazole obtained using the above process is characterized by any of its X-ray powder diffraction (“XRPD”) patterns, infrared absorption (“IR”) spectra, differential scanning calorimetry (“DSC”) curve, and thermogravimetric analysis (TGA) curve.

Crystalline Form B of omeprazole is characterized by its XRPD pattern. All XRPD data reported herein were obtained using Cu Kα radiation, having the wavelength 1.541 Å, and were obtained using a Bruker Axe, D8 Advance Powder X-ray Diffractometer.

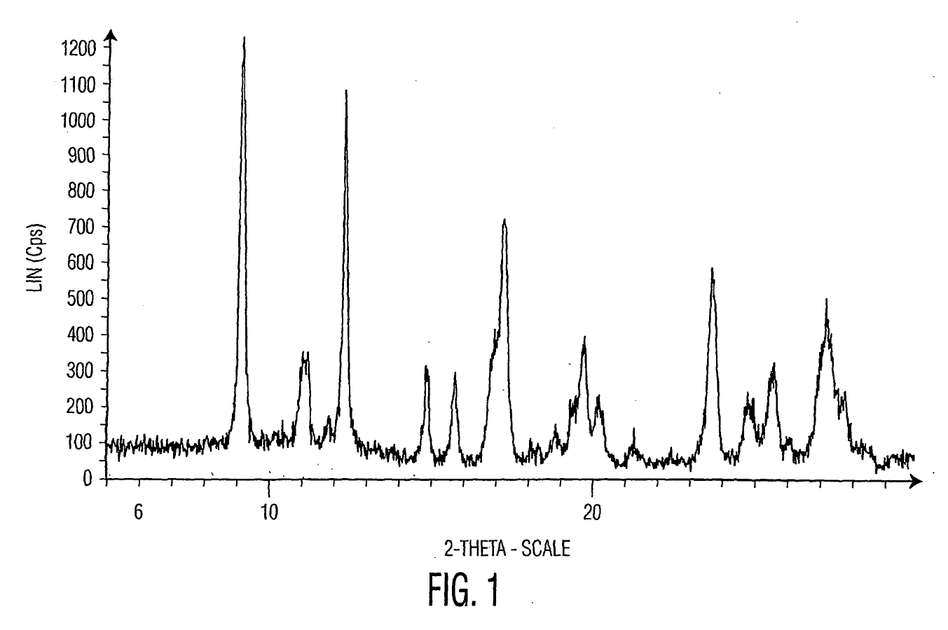

Crystalline Form B of omeprazole is characterized by its XRPD pattern substantially in accordance with the pattern of FIG. 1. The crystalline Form B of omeprazole is also characterized by an XRPD pattern having significant peaks at about 9.7, 8.0, 7.9, 7.2, 6.0, 5.6, 5.2, 5.1, and 4.5, ±0.2 degrees 2θ. It is also characterized by additional XRPD peaks at about 4.4, 4.3, 3.7, 3.5, 3.4, 3.2, and 3.0, ±0.2 degrees 2θ.

The infrared spectra of the stable crystalline Form B of omeprazole has been recorded on Perkin Elmer System 200 FT-IR spectrophotometers, between 400 cm⁻¹ and 4000 cm⁻¹, with a resolution of 4 cm⁻¹, in a potassium bromide pellet, the test compound being at the concentration of 1% by mass.

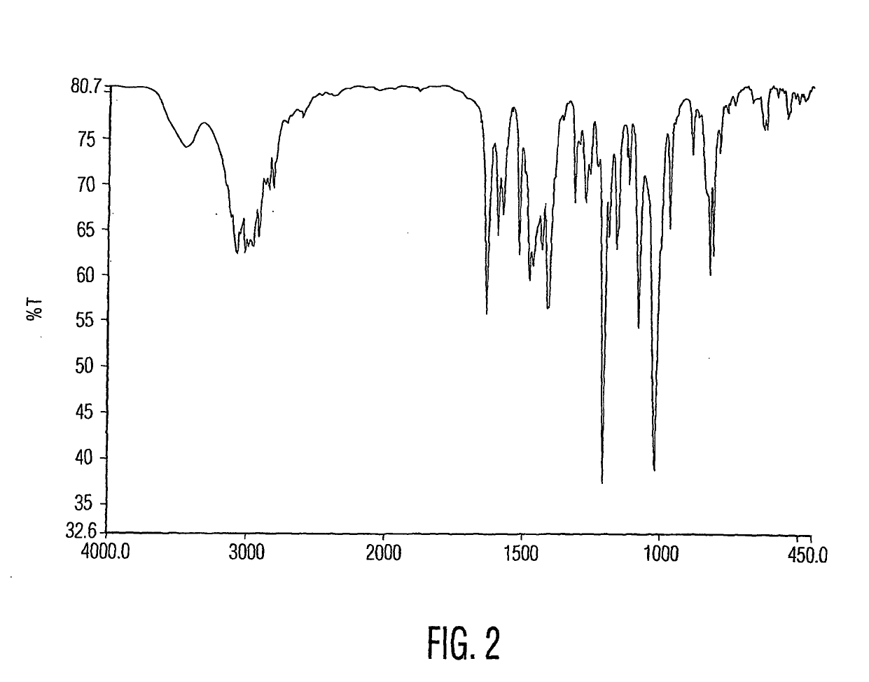

The crystalline Form B of omeprazole is characterized by an infrared absorption spectrum in potassium bromide comprising peaks at about 545, 620, 810, 821, 1017, 1077, 1204, 1407, 1627, 1587, 2802, 3006, and 3061, ±5 cm⁻¹. Crystalline Form B of omeprazole is also characterized by an infrared absorption spectrum substantially in accordance with the spectrum of FIG. 2.

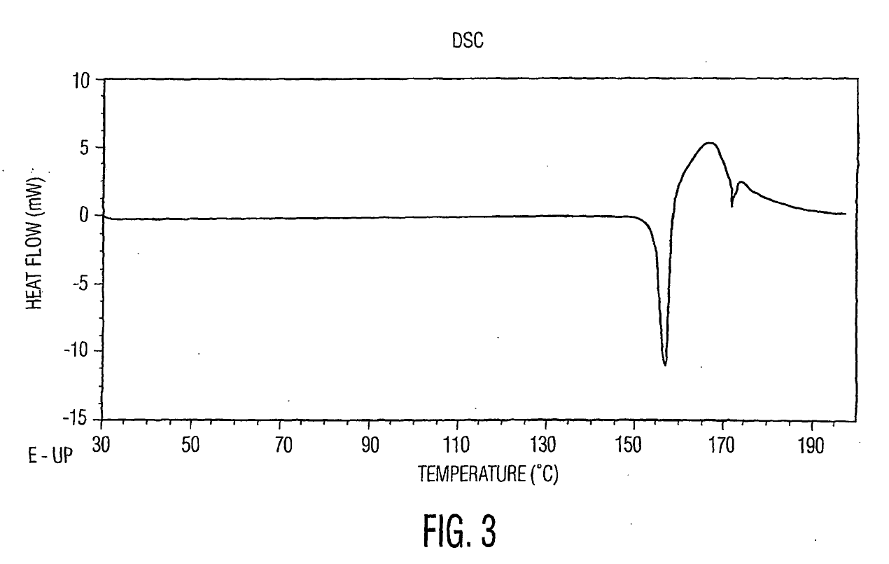

Differential scanning calorimetric analysis was carried out in a DSC Q1000 model from TA Instruments with a ramp of 5° C./minute with a modulation time of 60 seconds and a modulation temperature of ±1° C. The starting temperature was 0° C. and ending temperature was 200° C. Crystalline Form B of omeprazole is characterized by a differential scanning calorimetry curve having an endotherm at about 157° C. Crystalline Form B of omeprazole is also characterized by a differential scanning calorimetry curve substantially in accordance with FIG. 3,

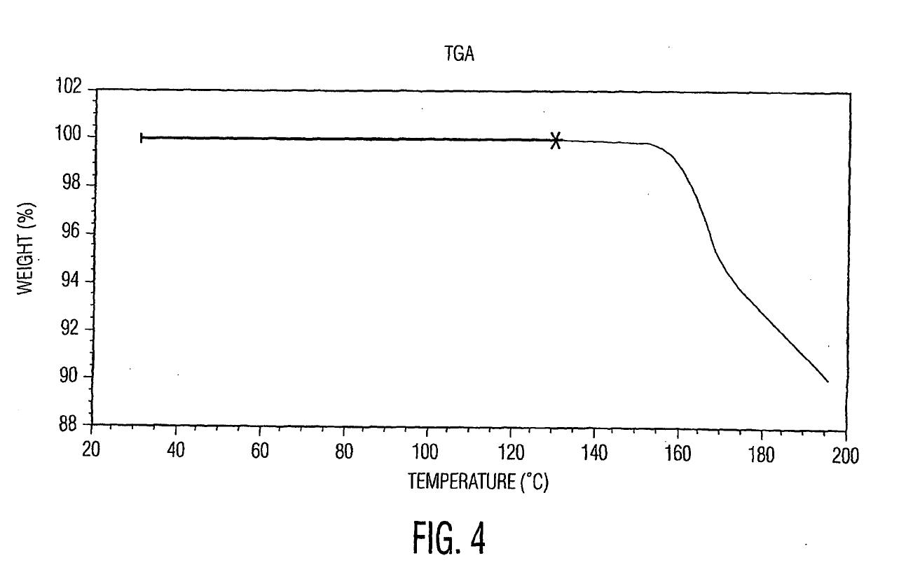

Crystalline Form B of omeprazole is further characterized by a thermogravimetric analysis curve substantially in accordance with the curve of FIG. 4, showing a loss of only 0.1% by weight.

The D₁₀, D₅₀ and D₉₀ values are useful ways for indicating a particle size distribution. D₉₀ refers to the value for the particle size for which at least 90 volume percent of the particles have a size smaller than the said value. Likewise D₅₀ and D₁₀ refer to the values for the particle size for which 50 volume percent, and 10 volume percent of the particles have a size smaller than the said value. A D₅₀ value can be considered as being the mean particle size of a powder. Methods for determining D₁₀, D₅₀ and D₉₀ include laser diffraction using Malvern equipment.

Crystalline Form B of omeprazole according to the invention has a D₁₀ less than about 5 μm or less than about 10 μm, D₅₀ less than about 10 μm or less than about 20 μm, and D₉₀ less than about 25 μm or less than about 50 μm. There is no specific lower limit for any of the D values.

Another aspect of the present invention provides a pharmaceutical composition comprising omeprazole Form B and at least one pharmaceutically acceptable excipient. Such pharmaceutical composition may be administered to a mammalian patient in a dosage form. The said compositions can be administered to human or animal orally, parenterally, rectally or by other such routes of administration.

Solid dosage forms for oral administration include tablet, capsule, pills, powder, granules, pellets or spheres comprising active substance, pellets or spheres coated with active substance, coated beads, coated microparticles, coated granules and the like.

The pellets or “cores” that can be used include but are not limited to water-soluble cores such as sugar spheres, lactose and the like; and water-insoluble cores such as microcrystalline cellulose, silicon dioxide, calcium carbonate, dicalcium phosphate anhydrous, dicalcium phosphate monohydrate, tribasic calcium phosphate, magnesium carbonate, magnesium oxide and the like.

The said solid dosage form can be prepared by mixing omeprazole Form B with pharmaceutically acceptable excipients like diluents, binders, glidants, disintegrants, pH adjusting agents, lubricants, colorants, flavoring agents, surfactants, anti-tack agents, opacifying agents and the like. The compositions may be immediate release or modified release like controlled release or delayed release forms where the release of the active substance can be modified by use of suitable release modifying excipients.

The pharmaceutical compositions of present invention can contain one or more diluents added to make the tablet or capsule mass making it easy to formulate and handle. Common diluents include, but are not limited to microcrystalline cellulose, powdered cellulose, lactose, starch, pregelatinized starch, calcium carbonate, calcium sulfate, sugar, dextrates, dextrin, dextrose, dibasic calcium phosphate dihyd rate, tribasic calcium phosphate, kaolin, magnesium carbonate, magnesium oxide, maltodextrin, mannitol, and the like.

Binders can also be included in the pharmaceutical compositions of the present invention to help hold granules or tablets together. Some examples include but are not limited to acacia, alginic acid, sodium alginate, gelatin, guar gum, starch, pregelatinized starch, polyvinyl pyrollidone, carboxymethyl cellulose, methyl cellulose, ethyl cellulose, hydroxyethyl cellulose, hydroxypropyl cellulose, hydroxypropyl methylcellulose, liquid glucose, magnesium aluminium silicate, dextrin, maltodextrin and polymethacrylates.

Lubricant such as talc, stearic acid, magnesium stearate, calcium stearate, sodium stearyl fumarate, colloidal silicon dioxide, glyceryl monostearate and the like can be included in the compositions of present invention.

Useful pH adjusting agents include but are not limited to alkalizers like sodium carbonate, potassium carbonate, magnesium carbonate, calcium carbonate, sodium hydroxide, magnesium hydroxide, aluminium magnesium hydroxide, magnesium metasilicate aluminate, magnesium silicate aluminate, sodium citrate, potassium citrate, sodium acetate, potassium acetate, organic bases like TRIS (tris (hydroxymethyl) aminomethane), arginine, tromethamine and meglumine; acidifiers like ascorbic acid, citric acid, tartaric acid, fumaric acid, acetic acid, malic acid, maleic acid, formic acid, propionic acid and hydrochloric acid.

Useful wetting agents include but are not limited to gelatin, casein, lecithin, gum acacia, cholesterol, tragacanth, stearic acid, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., TWEEN™), polyethylene glycols, polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxy propylcellulose, hydroxypropylmethylcellulose phthlate, magnesium aluminum silicate, triethanolamine, polyvinyl alcohol and polyvinylpyrrolidone (PVP), poloxamers such as PLURONIC™ F68, F127, and F108 which are block copolymers of ethylene oxide and propylene oxide and polyxamines such as TETRONIC™ 908.

The said compositions in the form of tablets or capsules can further include a disintegrant to accelerate disintegration of the said tablet or capsule. Useful disintegrants include but are not limited to alginic acid, carboxymethyl cellulose calcium, carboxymethyl cellulose sodium, colloidal silicon dioxide, crospovidone, guar gum, magnesium aluminium silicate, microcrystalline cellulose, powdered cellulose, polacrillin potassium, sodium alginate, sodium starch glycolate, starch and pregelatinized starch.

The compositions can be prepared using processes known to one skilled in the art, such as direct compression, dry granulation, compaction granulation or wet granulation.

The compositions can be further coated. The coating may be seal coating, film coating, subcoating, barrier coating, polishing coating, compression coating, fast disintegrating coating, enzyme degradable coating, sugar coating, release-modifying coating like polymeric or enteric coat, specialized coatings like bioadhesive coatings, and such the like, or combinations thereof. Coating can be achieved by methods such as by using fluidized bed equipment, perforated pans, a regular pharmaceutical pan, compression coating, continuous or short spray methods, by drenching or such other methods known to one skilled in the art.

Further, multiple coatings can be applied to achieve desired results. The active substance can be present in the core or coat(s) or both. The cores in the form of pellets, granules or tablets may be coated with active substance along with pharmaceutically acceptable excipient(s) like alkalizers and the cores can be optionally coated with release modifying or specialized coating. There can be a separate coating of alkalizer or it can be present in any of the outer coating layers. The coatings can comprise other excipients like plasticizers such as triethyl citrate, acetyl triethyl citrate, acetyl tributyl citrate, acetylated monoglycerides, glycerin, triacetin, propylene glycol, phthalate esters, castor oil, sorbitol and dibutyl sebacate; anti-tackiness agents like talc and glyceryl monostearate; pigments like titanium dioxide or ferric oxides, polishing agents like glyceryl monostearate, carnauba wax, candellila wax and the like.

The solvents that may be used for preparation of coating composition may be organic or inorganic, including water, or mixtures thereof. Organic solvents like methanol, ethanol, acetone, dichloromethane, isopropanol and the like can be used in the present invention.

The excipients that may be used to modify the release of active substance in the present invention can be hydrophilic or hydrophobic or combinations of both. The hydrophobic release modifying agents can be selected from include but are not limited to ethyl cellulose, cellulose acetate, cellulose propionate, cellulose acetate propionate, cellulose acetate butyrate, cellulose triacetate, methacrylic acid copolymer type A, B and C, polymeric acrylate derivatives, pluronic block copolymers, polyvinyl acetate, waxes like beeswax, carnauba wax, microcrystalline wax and ozokerite, fatty alcohols like cetostearyl alcohol, stearyl alcohol, cetyl alcohol and myristyl alcohol, fatty acid esters like glyceryl monostearate, glycerol distearate, glycerol monooleate, acetylated monglyceride, tristearin, tripalmitin, palmitostearate, glyceryl behenate and hydrogenated castor oil.

The hydrophilic release modifying agents can be selected from include but are not limited to hydroxymethyl cellulose, hydroxyethyl cellulose, hydroxypropyl cellulose, hydroxybutyl cellulose, hydroxyethyl methyl cellulose, hydroxypropyl methyl cellulose, carboxymethylcellulose, sodium carboxymethylcellulose, carboxymethyl ethyl cellulose, carboxyalkylcellulose esters, natural, semisynthetic or synthetic polysaccharides such as alginic acid, alkali metal and ammonium salts, carrageenans, galactomannans, tragacanth, agar-agar, gum arabic, guar gum, xanthan gum, pectins like sodium carboxymethylamylopectin, chitosan, polyfructans, inulin, polyacrylic acids, polymethacrylic acids, methacrylate copolymers, polyvinyl alcohol; polyvinylpyrrolidone, copolymers of polyvinylpyrrolidone with vinyl acetate, combinations of polyvinyl alcohol and polyvinylpyrrolidone, polyalkylene oxides such as polyethylene oxide and polypropylene oxide and copolymers of ethylene oxide and propylene oxide.

The enteric excipients can include but are not limited to methacrylic acid copolymers type A, B and C, hydroxypropyl methylcellulose phthalate, hydroxypropyl methylcellulose acetate succinate, cellulose acetyl phthalate, cellulose diacetyl phthalate, cellulose triacetyl phthalate, cellulose acetate phthalate, polyvinyl acetate phthalate, sodium cellulose acetate phthalate, cellulose ester phthalate, cellulose ether phthalate, methylcellulose phthalate, cellulose ester-ether phthalate, hydroxypropyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate diethyl phthalate, dibutyl phthalate, dialkyl phthalate, cellulose acetate trimelliate, shellac, zein and mixtures thereof.

In an embodiment, a pharmaceutical composition comprises a pharmaceutically inert pellet coated with omeprazole Form B, and having an exterior enteric coating.

In one embodiment of the present invention, compositions of the omeprazole

Form B comprise:
a) cores comprising pharmaceutically inert substances;
b) seal coating of the inert cores using an aqueous dispersion or solution of one or more polymeric substances;
c) layering of seal coated cores with a composition comprising omeprazole with or with out other pharmaceutically acceptable excipients as aqueous or non-aqueous dispersion or solution over the coated cores;
d) sub-coating over the drug layered cores using an aqueous or non-aqueous dispersion or solution of one or more polymeric substances with or with out other pharmaceutically acceptable excipients; and
e) enteric coating over the sub-coated cores using an aqueous or non-aqueous dispersion or solution of one or more enteric-coated substances with or with out other pharmaceutically acceptable excipients.

The enteric-coated cores can be blended with other pharmaceutically acceptable excipients and further compressed as tablets, or filled into capsules.

In another embodiment, the compositions of omeprazole Form B comprise:
a) cores of a pharmaceutically inert substance like sugar spheres;
b) seal coating of the inert spheres using an aqueous solution of hypromellose;
c) layering of seal coated spheres with a suspension of omeprazole Form B in hydroxypropyl methylcellulose and an alkalizer;
d) subcoating the drug layered spheres with a suspension of hydroxypropyl methylcellulose and opacifier;

e) enteric coating of the subcoated spheres using a solution comprising a methacrylate copolymer like EUDRAGIT™ L 100 55; and filling the enteric-coated spheres into gelatin capsule shells.

The process of our invention is advantageous because of the stable product obtained. The solvents used are inexpensive and easily regenerated. No impurities are produced in the process. The product obtained can be used for preparing pharmaceutical formulations.

These and other specific aspects and embodiments of the invention are described in further detail by the examples below, which examples are not intended to limit the scope of the appended claims in any manner.

### Example 1

### Preparation of Pure Crystalline Form B of Omeprazole

90 liters of dichloromethane and 24 liters of methanol were taken into a reactor and the solvent mixture was cooled to 4° C. Omeprazole was charged into the reactor at 4° C. and the reaction mass was stirred for 10 minutes. The reaction mass was checked for clear dissolution. Then 24 liters of demineralized water was added to the reaction mass at 3° C. The reaction mass was stirred for 10 minutes at the same temperature. Then the reaction mass from the reactor was transferred into the feed tank. The reaction mass was cooled to 2° C. The cooled reaction mass was fed into an ATFD (manufactured by: Techno Force (I) Pvt. Ltd, ASME SEC; VIII Div. I ED-2001+A03) with a feed rate of 45-65 liters/hour at a jacket temperature of 37° C. After completion of the feeding, the ATFD was run for another 3 to 5 minutes to remove solvent traces. The material was unloaded. The wet material was initially dried in a vacuum tray dryer at a temperature of 54° C. and a vacuum of 700 mm Hg for about 3 hours 30 minutes to get a semi-dried material. The semi-dried material was micronised in a microniser. The feed rate of the semi dried material into the microniser was adjusted to 2 to 3 kg/hour under an air pressure of 4 kg/cm². The micronised material was re-dried in a vacuum tray drier at a temperature of 53° C. and a vacuum of 700 mm Hg for 8 hours, 30 minutes. Finally, the dried material was sifted through a 40 mesh sieve to yield 10.3 kg of the title compound.

Particle Size Distribution: D₁₀: 0.77 μm, D₅₀: 3.20 μm, D₉₀: 7.15 μm.

### Example 2

### Stability Study of Omeprazole Form B

Omeprazole Form B prepared using a method similar to the one described in Example 1 was subjected to long-term stability studies. Each sample was packed in a clear polythene bag, filled with nitrogen, tied with a tag and kept in a black polythene bag along with silica pouch filled with nitrogen and then sealed. Finally, the above triple laminated bag along with silica pouch was sealed and stored in a HDPE drum. The color, moisture content, purity and the polymorphic form of the product was checked at intervals during storage. The results for compliance of polymorphic form are tabulated below, where “ND” means not detected by XRPD.

Form A (%)

| Storage Conditions | Initial | 3 Months | 6 Months |
|---|---|---|---|
| Temperature 25 ± 2° C. | ND | ND | ND |
| Relative Humidity: 60 ± 5% | | | |
| Temperature 2 to 8° C. | ND | ND | ND |
| Relative Humidity: 60 ± 5% | | | |
| Temperature 40 ± 2° C. | ND | ND | ND |
| Relative Humidity: 60 ± 5% | | | |

The color, moisture content and purity of the product also remained substantially the same during the storage.

### Example 3

### Determination of Omeprazole Form a in Form B by X-Ray Diffraction

### Standard Preparation:

950 mg of Form B and 50 mg of Form A of Omeprazole were weighed and combined to form a weight ratio of Form B to Form A of 95:5. The mixture was ground three times in a mortar to get a uniform powder.

Instrument Conditions

| | |
|---|---|
| Make, Model | Bruker AXS, D8 Advance |
| | powder X-ray diffractometer |
| Goniometer | Theta/Theta vertical |
| Measuring circle | 435 mm |
| Radiation | Cu K alpha-1 (Wavelength = 1.5406 A) |
| Tube | 2.2 kW copper long fine focus |
| Detector | Scintillation counter. |
| Voltage, Current | 40 kV, 50 mA |
| Scan type | Locked coupled |
| Scan mode | Step scan |
| Divergence slit | 1.0 deg. |
| Antiscattering slit | 1.0 deg. |
| Detector slit | 0.2 mm |
| Synchronuous rotation | On |
| Scan range | 19.9° to 21.0° 2θ |
| Step size | 0.01 deg. |
| Time/Step | 25.0 sec. |

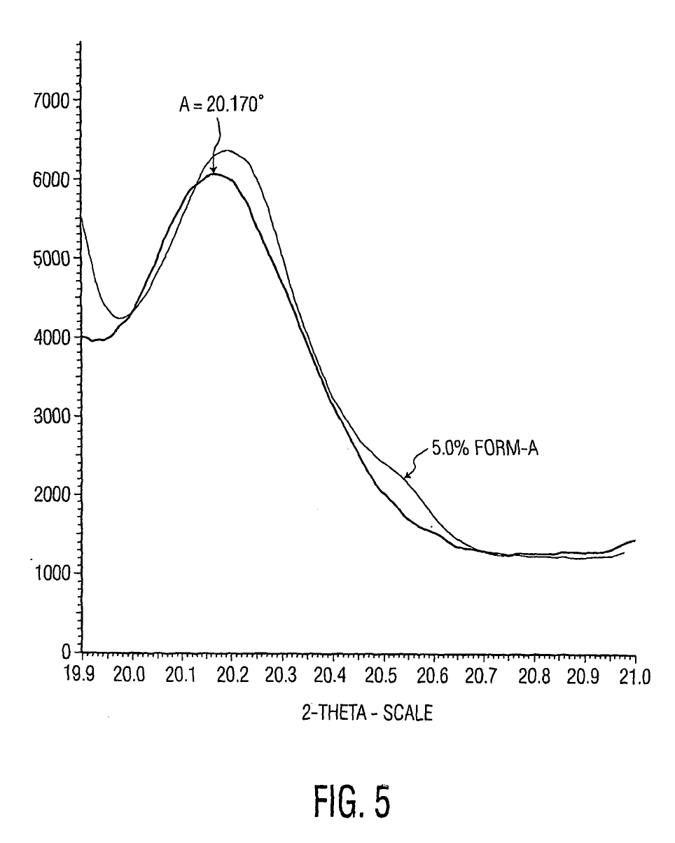

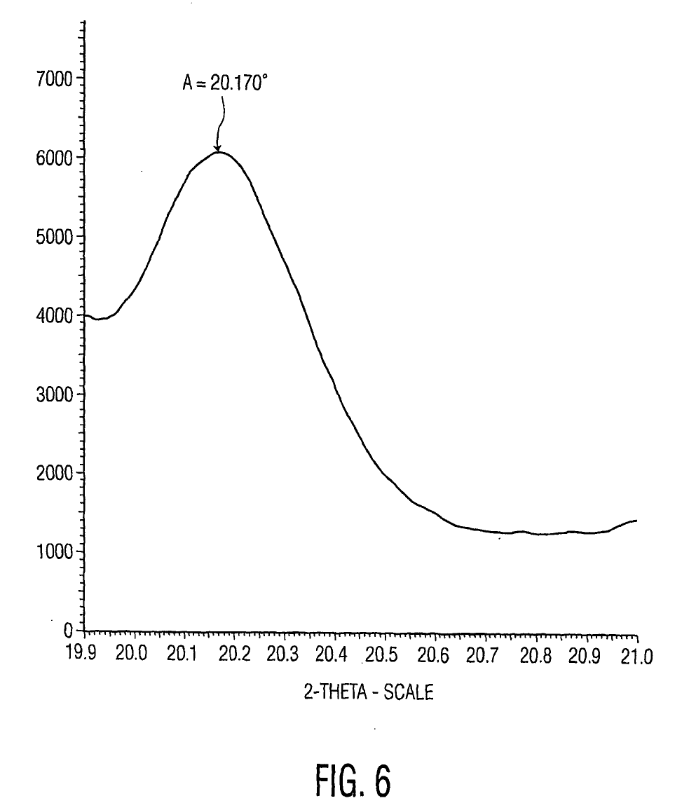

FIG. 6 is a scan for pure omeprazole Form B. FIG. 5 is the XRPD scan for the standard, containing 5% by weight of omeprazole Form A, and the scan of FIG. 6 has been superimposed for comparison.

### Example 4

### Composition Comprising Omeprazole Form B

| | |
|---|---|
| Ingredient | mg/Capsule |

Seal Coating

| | |
|---|---|
| Sugar pellets | 145 |
| Hydroxypropyl methylcellulose (5 cps) | 7.5 |
| Water | 124 |

Drug Coating

| | |
|---|---|
| Omeprazole Form B | 40 |
| Hydroxypropyl methylcellulose (5 cps) | 10 |
| Meglumine | 5 |
| Poloxamer 407* | 5 |
| Water | 340 |

Subcoating

| | |
|---|---|
| Hydroxypropyl methylcellulose (5 cps) | 24.6 |
| Talc | 3.7 |
| Titanium dioxide | 3.7 |
| Water | 414 |

Enteric Coating

| | |
|---|---|
| Eudragit L 100 55** | 37.7 |
| Triethyl citrate | 7.5 |
| Talc | 3.8 |
| Isopropyl alcohol | 1125 |

Polishing Coat

| | |
|---|---|
| Glyceryl monostearate | 0.25 |
| Talc | 0.5 |
| Isopropyl alcohol | 15 |

*Poloxamer 407 is poly(ethylene oxide)-poly(propylene oxide)-poly(ethylene oxide) amphiphilic block copolymer.
**Methacrylic acid copolymer (Type C)

### Manufacturing Process:

### Seal Coating:

1. Hydroxypropyl methylcellulose was dissolved in water.
2. The above solution was coated onto sugar pellets in a fluid bed processor at an inlet air temperature of 60° C.

### Drug Coating:

3. Ingredients 5, 6 and 7 were dissolved in water and omeprazole was suspended in this solution.
4. The seal coated pellets of step 2 were coated with the suspension of step 3 in a fluid bed coater at an inlet temperature of 55° C.

### Subcoating:

5. Ingredient 9 was dissolved in water.
6. Ingredients 10 and 11 were suspended in solution of step 5.
7. The pellets of step 4 were coated with the suspension of step 6 in a fluid bed coater at an inlet temperature of 60° C.

### Enteric Coating:

8. Eudragit L 100 55 was dissolved in isopropyl alcohol.
9. Triethyl citrate was added to the solution of step 7.
10. Talc was added to it and stirred for 20 minutes.
11. The pellets of step 6 were coated with suspension of step 9 in a fluid bed coater at an inlet temperature of 45° C.

### Polishing Coat:

12. Glyceryl monostearate and talc were added to isopropyl alcohol and homogenized for 15 minutes.
13. The enteric-coated pellets of step 9 were coated with the polishing coating suspension of step 11 in a fluid bed coater at an inlet temperature of 45° C.
14. The pellets of step 13 were filled into size 1 hard gelatin capsule shells.

### Example 5

### Dissolution Study of the Composition of Example 4

Apparatus: USP I (basket)

Medium: 900 ml, pH 6.8 phosphate buffer.

Stirring speed: 100 rpm

Drug Release (%)

| Time | Composition of | Prilosec ® 40 mg DR |
|---|---|---|
| (min) | Example 3 | Capsule |
| 10 | 81 | 84 |
| 20 | 96 | 92 |
| 30 | 96 | 93 |
| 45 | 94 | 92 |

### Example 6

### Composition Comprising Omeprazole Form B without Seal Coating

| | |
|---|---|
| Ingredient | mg/capsule |

Drug Coating

| | |
|---|---|
| Sugar pellets | 150 |
| Omeprazole Form B | 40 |
| Hydroxypropyl methylcellulose (5 cps) | 10 |
| Meglumine | 5 |
| Poloxamer 407 | 5 |
| Water | 187 |

Subcoating

| | |
|---|---|
| Hydroxypropyl methylcellulose (5 cps) | 25.4 |
| Talc | 3.8 |
| Titanium dioxide | 3.8 |
| Water | 555 |

Enteric Coating

| | |
|---|---|
| Eudragit L 100 55 | 44.1 |
| Triethyl citrate | 4.4 |
| Magnesium stearate | 2.5 |
| Isopropyl alcohol | 555 |

### Manufacturing Process:

### Drug Coating:

1. Ingredients 2, 3, 4 and 5 were suspended in water
2. The sugar pellets were coated with the suspension of step 1 in a fluid bed coater at an inlet temperature of 60° C.

### Subcoating:

3. Ingredients 7, 8 and 9 were suspended in water.
4. The pellets of step 3 were coated with the suspension of step 3 in a fluid bed coater at an inlet temperature of 60° C.

### Enteric Coating:

5. Eudragit L 100 55 was dissolved in isopropyl alcohol.
6. Triethyl citrate was added to the solution of step 5.
7. Magnesium stearate was added to it and stirred for 20 minutes.
8. The pellets of step 4 were coated with suspension of step 6 in a fluid bed coater at an inlet temperature of 45° C.
9. The pellets of step 8 were filled into size 1 hard gelatin capsule shells.

### Example 7

### Composition Comprising Omeprazole Form B without Seal Coating

| | |
|---|---|
| Ingredient | mg/Capsule |

Drug Coating

| | |
|---|---|
| Sugar pellets | 130 |
| Omeprazole Form B | 40 |
| Hydroxypropyl methylcellulose (5 cps) | 20 |
| Polyoxyethylene (20) sorbitan monooleate | 1 |
| Water | 100 |

Subcoating

| | |
|---|---|
| Hydroxypropyl methylcellulose (5 cps) | 19.3 |
| Talc | 5.8 |
| Titanium dioxide | 3.9 |
| Water | 165 |

Enteric Coating

| | |
|---|---|
| Hydroxypropyl methylcellulose phthalate | 39.1 |
| Cetyl alcohol | 2 |
| Titanium dioxide | 2.9 |
| Acetone | 198 |
| Water | 198 |

### Manufacturing Process:

### Drug Coating:

1. Ingredients 3 and 4 were dissolved in water. Omeprazole was suspended in this solution.
2. The sugar pellets were coated with the suspension of step 1 in a fluid bed coater at an inlet temperature of 55° C.

### Subcoating:

3. Ingredients 6, 7 and 8 were suspended in water.
4. The pellets of step 3 were coated with the suspension of step 3 in a fluid bed coater at an inlet temperature of 60° C.

### Enteric Coating:

5. Hydroxypropyl methylcellulose phthalate and cetyl alcohol were dissolved in acetone:water (90:10) mixture.
6. Titanium dioxide was added to the solution of step 5.
7. The pellets of step 4 were coated with suspension of step 6 in a fluid bed coater at an inlet temperature of 45° C.
   The pellets of step 7 were filled into size 1 hard gelatin capsule shells.

1. A process for preparing omeprazole crystalline Form B, comprising removing solvents from a solution of omeprazole in a mixture comprising a halogenated hydrocarbon and an alcohol.

2. The process of claim 1, wherein a halogenated hydrocarbon comprises dichloromethane.

3. The process of claim 1, wherein an alcohol comprises methanol.

4. The process of claim 1, wherein a halogenated hydrocarbon comprises dichloromethane and an alcohol comprises methanol.

5. The process of claim 4, wherein a ratio of dichloromethane to methanol is about 4:1.

6. The process of claim 1, wherein a solution of omeprazole is washed with water, prior to solvent removal.

7. The process of claim 6, wherein a solution is washed with water in an amount about 1 to 5 times the weight of omeprazole present.

8. The process of claim 1, wherein solvent removal occurs in equipment that provides flash evaporation.

9. The process of claim 1, wherein solvent removal occurs in an agitated thin film drier.

10. The process of claim 1, further comprising drying an omeprazole crystalline Form B product.

11. Omeprazole crystalline Form B prepared by the process of claim 1 and having D₁₀ less than about 10 μm, D₅₀ less than about 20 μm, and D₉₀ less than about 50 μm.

12. A composition comprising omeprazole crystalline Form B prepared by the process of claim 1, coated onto a pharmaceutically inert core.

13. The composition of claim 12, wherein a pharmaceutically inert core comprises sugar.

14. The composition of claim 12, comprising a coating of a hydrophilic or hydrophobic release modifier over an inert core having an omeprazole coating.

15. The composition of claim 12, comprising an outer enteric coating.

16. A composition comprising multiple coated cores of claim 12.

17. A composition comprising multiple coated cores of claim 15.
